(19) United States
(12) Patent Application Publication
(10) Pub. No.: US 2026/0276757 A1
(43) Pub. Date: Sep. 17, 2026

(54) OPTICALLY PUMPED MAGNETOMETER CELL

(71) Applicant: MAG4HEALTH, GRENOBLE (FR)

(72) Inventor: Agustin PALACIOS-LALOY, GRENOBLE (FR)

(21) Appl. No.: 19/164,417

(22) PCT Filed: Mar. 19, 2024
(86) PCT No.: PCT/EP2024/057329

§ 371 Date: Sep. 11, 2025

(30) Foreign Application Priority Data
Mar. 24, 2023 (FR) FR2302837

(51) Int. Cl. G01R 33/26; A61B 5/00

(52) U.S. Cl. CPC G01R 33/26; A61B 5/6803

(57) ABSTRACT

The invention relates to a cell for an optically pumped magnetometer, including: —an enclosure containing a gas; and —a circuit for exciting a plasma in the enclosure, which comprises one or more elements (11) made of an electrically conductive material arranged so as to apply an electrical discharge to the gas enclosed in the enclosure. A conductance of the excitation circuit is less than 8000 Siemens. This conductance is defined as the sum of the conductance of each of the element or elements made of electrically conductive material wherein the conductance of an element made of electrically conductive material corresponds to the conductivity of the electrically conductive material weighted by the ratio between the volume of electrically conductive material of the element and the square of the distance between the element and the centre of the cell.

### TECHNICAL FIELD

The field of the invention is that of optically pumped magnetometers.

### PRIOR ART

Optically pumped magnetometers use atomic gases confined in a cell, typically metastable helium or alkaline gases, as the sensing element. These magnetometers, which can be configured in various ways, enable the magnetic field to be determined by exploiting the following three processes, which take place either sequentially or concomitantly:

1) Using polarised light sources, typically lasers, enables atomic states to be produced which are characterised by a certain orientation or alignment of their spins. This process is known in the field as “optical pumping”.

2) These atomic states change under the effect of the magnetic field, in particular under the Zeeman effect which corresponds to shifts in energy levels as a function of the magnetic field to which the atoms are subjected.

3) The optical properties of the atomic medium then undergo changes that depend on the state of the atoms. An optical measurement, for example an optical absorption measurement, can then be used to determine the Zeeman shift experienced, and to deduce a measurement of the magnetic field in which the cell is immersed.

The sensitivity, also known as intrinsic low noise, achievable with such optically pumped magnetometers is remarkable and significantly better than that of most other magnetic measurement technologies (fluxgate, Hall effect, magnetoresistance, etc.). Only the SQUID magnetometer has a similar noise, but it requires cryogenic cooling of the sensitive element, which contains elements that need to be superconducting for its operation, which restricts its practical scope of application.

Measurement of magnetic fields is useful for various applications, in particular the characterisation of electrical currents circulating in the human body, making it possible, for example, to understand and diagnose various brain and heart pathologies. In such a use for measurements on the human body, it is advantageous not to have a single magnetometer, but a dense network of magnetometers providing good spatial resolution. This requires the magnetometers to be of sufficiently small lateral size (usually referred to as “miniature” magnetometers). Since these biomagnetic imaging techniques involve imaging the inside of the human body, where the magnetic sources are typically one to a few centimetres from the surface of the body, it is ideal to have magnetometers of a size similar to this distance, typically with a lateral size of between 3 mm and 3 cm.

Optically pumped alkali magnetometers of centimetre lateral size that are compatible with networking now achieve sensitivities close to 10 fT/sqrt (Hz). This sensitivity can be understood as the “intrinsic noise” that the operation of the magnetometer adds to the measured signal. It arises from various phenomena, in particular the noise of the light used to measure atomic states, which, in the best case, exhibits quantum fluctuations also known as “photon noise” or “optical shot noise”.

In the case of optically pumped metastable helium magnetometers, sensitivities of the order of 50 fT/sqrt (Hz) were reported in the article [1] listed below. Such sensitivity is not as favourable as in the case of alkali magnetometers. However, optically pumped metastable helium magnetometers have many other practical advantages. In particular, they do not require heating in order to operate. They also have a much broader bandwidth.

### DISCLOSURE OF THE INVENTION

The aim of the invention is to reduce the intrinsic noise of optically pumped metastable helium magnetometers.

For this purpose, the invention proposes a cell for an optically pumped magnetometer, comprising:
an enclosure containing a gas; and
a circuit for exciting a plasma in the enclosure, which comprises one or more elements of electrically conductive material arranged with respect to the enclosure so as to enable an electrical discharge to be applied to the gas contained in the enclosure.

A conductance of the excitation circuit is defined as the sum of the conductance of each of the one or more elements made of electrically conductive material, where the conductance of an element made of electrically conductive material corresponds to the conductivity of the electrically conductive material weighted by the ratio between the volume of electrically conductive material of said element and the square of the distance between said element and the centre of the cell. According to the invention, the conductance of the excitation circuit is less than 8000 Siemens, preferably less than 5000 Siemens, even more preferably less than 1500 Siemens.

Some preferred but non-limiting aspects of this cell are as follows:
the enclosure has a characteristic dimension of between 3 mm and 3 cm, preferably between 5 mm and 2 cm;
the excitation circuit is capacitively coupled to the cell, the element or elements of electrically conductive material consisting of two electrodes;
each of the electrodes consists of a ring of electrically conductive material which surrounds the enclosure;
each of the electrodes consists of a deposit of electrically conductive material on the enclosure;
each of the electrodes consists of a print of electrically conductive material on a dielectric film attached to the enclosure;
the dielectric film is flexible;
the excitation circuit is inductively coupled to the enclosure, the element or elements of electrically conductive material consisting of a coil wound in a plurality of loops around the cell;
the coil is a solenoid;
the coil is a helix of a helical resonator.

The invention also relates to an optically pumped magnetometer comprising a cell according to the invention and a magnetoencephalography helmet comprising a plurality of magnetometers according to the invention.

### BRIEF DESCRIPTION OF THE FIGURES

Other aspects, aims, advantages and features of the invention will become clearer from the following detailed description of preferred embodiments thereof, given by way of non-limiting example, and made with reference to the appended drawings in which:

FIG. 1 shows a diagram of a magnetometer incorporating a cell in accordance with the invention;

FIG. 2 is an example of an electrode deposited on a flexible dielectric film in a fork pattern.

### DETAILED DISCLOSURE OF SPECIFIC EMBODIMENTS

Unlike optically pumped alkali magnetometers, optically pumped metastable helium magnetometers are based on an atomic state that is not the ground state (1¹S₀ in spectroscopic notation for helium-4), but an excited state, in this case the metastable triplet state (denoted 2³S₁ for helium-4). This state must be populated in order for the magnetometer to function. This is usually achieved using an electrical discharge operating in the high frequency range, typically between 1 MHz and 100 MHz. This discharge creates a plasma inside the cell, in which high-energy collisions between atoms and electrons excite some of the atoms into a metastable state. Various strategies can be used to initiate and maintain this plasma discharge:
a capacitively coupled discharge circuit, consisting of two electrodes placed on either side of the gas cell; or
an inductively coupled discharge circuit, consisting of a solenoid located so that the magnetic flux it creates is partly applied to the gas located inside the cell. One variant consists of a helical resonator connected to a generator only at one of its ends and facing a ground plane. For certain frequencies, this resonator behaves like a solenoid, with advantages in terms of impedance matching to the generator.

For measurement applications on the human body, it is preferable for the magnetometer to be small. Ideally, the components of the magnetometer other than the sensitive element (in particular the capacitive or inductive element which enables the plasma to be ignited and maintained) should not take up much space and should be located in the immediate vicinity of this sensitive element, so that the overall dimensions of the sensor are only slightly greater than the dimensions of the sensitive element it contains. Capacitive electrodes are advantageous in this respect, as well as an inductive circuit comprising a coil with a diameter very slightly greater than that of the helium cell.

A study of the optimum discharge regimes for obtaining a high-amplitude magnetometry signal has been carried out and is published in the thesis of J. Rutkowski [2], for cells with characteristic dimensions of 5 mm and 1 cm and electrodes consisting of fragments of adhesive-backed copper tape.

Other capacitive electrodes are mentioned in the literature, in particular electrodes consisting of two (or more) rings which surround the cell. These electrodes are mentioned in the thesis of Marie-Constance Corsi [3], which mentions a wire that is “thicker” than the one used to connect the adhesive-backed copper tape (which is 0.1 mm). The patent application [4] associated with this work discloses a wire diameter of 315 microns.

In addition to obtaining a strong magnetometry signal, it is also desirable for the intrinsic noise of the magnetometer to be limited to its unavoidable components, in particular photon noise, which occurs when other technical noise (current noise in the laser, for example) becomes negligible. Since the photon noise can be calculated, the Applicant was able to ascertain that there was still technical noise, of an unknown nature, which limited the total noise of the sensor. The Applicant suspected that this technical noise was related to the electrodes, which is now proven as will be described below.

The Applicant has thus been able to show that in the case of sensors based on the optical pumping of metastable helium, the adhesive-backed copper tape electrodes traditionally used (thickness between 35 and 100 microns) and the thick wire loop electrodes also used in the past (thickness of 315 or 500 microns) introduce significant magnetic noise, which, for optimised sensors, becomes the limiting noise of the sensor.

Furthermore, the Applicant has found that it is possible to significantly reduce this noise contribution, thereby achieving more favourable levels of sensitivity than before, by drastically limiting the amount of conductive material that constitutes the means for exciting the plasma discharge (electrodes or inductive discharge circuit) placed close to the measurement cell.

Based on this observation, the Applicant proposes a cell for an optically pumped magnetometer equipped with a circuit for exciting a plasma in an enclosure containing an atomic gas by means of an electrical discharge, the circuit being optimised to create high densities of metastable atoms, with long lifetimes, without creating additional technical noise due to this discharge or its excitation means.

The invention thus relates to a cell for an optically pumped magnetometer, which comprises an enclosure containing a gas and a circuit for exciting a plasma in the enclosure. The magnetometer is preferably a miniature magnetometer, the enclosure then having a characteristic dimension (diameter if it is a sphere, side if it is a cube, diameter and height if it is a cylinder) of between 3 mm and 3 cm, preferably between 5 mm and 2 cm.

The gas may be helium-4.

The circuit for exciting a plasma in the enclosure comprises one or more elements of electrically conductive material arranged with respect to the enclosure so as to enable an electric discharge to be applied to the gas contained in the enclosure. This circuit is powered by an RF radio frequency generator, for example in the 10-100 MHz range.

According to the invention, the quantity of conductive material placed close to the sensitive element is limited so that the conductance of the excitation circuit is less than 8000 Siemens, preferably less than 5000 Siemens, even more preferably less than 1500 Siemens. By reducing this conductance to values of less than 8000 Siemens, it becomes possible to exceed the best sensitivities previously obtained on miniature helium magnetometers.

In the context of the invention, this conductance Qₜₒₜₐₗ of the excitation circuit is defined as the sum of the conductance Q of each of the one or more elements of electrically conductive material. In other words, when the excitation circuit has a single element of electrically conductive material, its conductance Qₜₒₜₐₗ corresponds to that of the element of electrically conductive material. Further, when the excitation circuit has several elements made of electrically conductive material, its conductance Qₜₒₜₐₗ corresponds to the sum of the individual conductance of each of the elements made of electrically conductive material.

The conductance of an element made of electrically conductive material corresponding to the conductivity σ of the electrically conductive material (in Siemens/m) weighted by the ratio between the volume V of electrically conductive material of said element (in m³) and the square of the distance a (in m) between said element and the centre of the cell, i.e.

$$Q = σ * \frac{V}{{a}^{2}} .$$

Thus, when the excitation circuit comprises several elements made of electrically conductive material (two electrodes, for example), its conductance Qₜₒₜₐₗ corresponds to the sum of the conductance Q of the various elements.

In one possible embodiment, the excitation circuit is capacitively coupled to the cell, the element or elements of electrically conductive material consisting of two electrodes.

Each of the electrodes may consist of a ring of electrically conductive material which surrounds the enclosure, in accordance with the arrangement disclosed in [3] but having a conductance in accordance with the invention.

In a first variant, each of the electrodes may consist of a deposit of electrically conductive material on the enclosure.

In a second variant, each of the electrodes may consist of a print of electrically conductive material on a dielectric film, this film being attached, for example by gluing, on the enclosure. The dielectric film is preferably a flexible film.

In another possible embodiment, the excitation circuit is inductively coupled to the enclosure, the element or elements of electrically conductive material consisting of a coil wound in a plurality of loops around the cell.

The coil may be a solenoid or may form the helix of a helical resonator.

The invention extends to an optically pumped magnetometer comprising a cell as previously described and to a magnetoencephalography helmet comprising a plurality of magnetometers according to the invention.

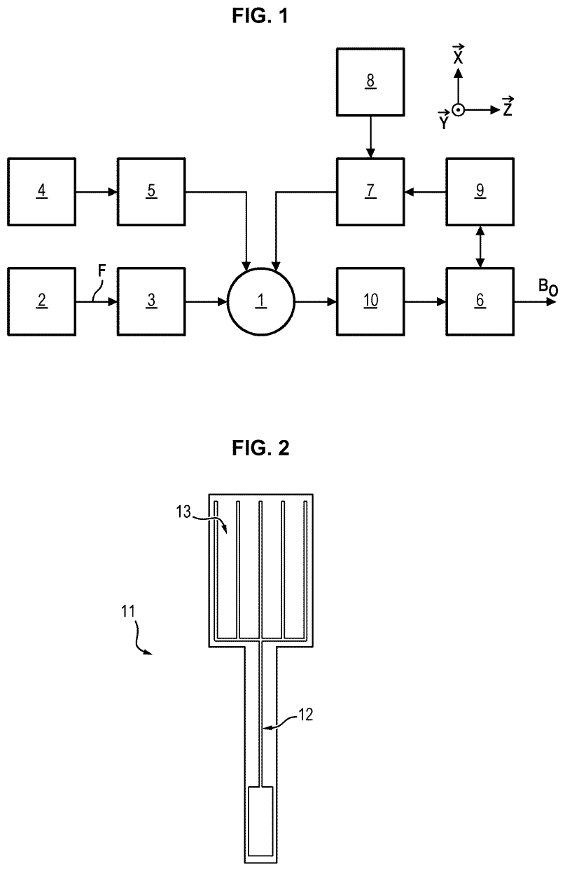

With reference to FIG. 1, such an optically pumped magnetometer has a cell comprising an enclosure 1 filled with an atomic gas, for example helium-4, subjected to an ambient magnetic field B0, the projection of which on three rectangular coordinate axes defines three components.

For example, such a magnetometer could be based on an enclosure filled with high-purity helium-4. This enclosure may have a characteristic dimension of between 3 mm and 3 cm (diameter if it is a sphere, side if it is a cube, diameter and height if it is a cylinder). It is filled with helium at a pressure that depends on its size, typically 10 torr for a 1 cm cylindrical cell.

The cell is illuminated by an optical pumping source 2 capable of emitting a beam of light F, for example a laser beam tuned to a pumping wavelength, towards the cell 1 (this beam is also referred to as a pump beam). The pumping wavelength is set to an atomic transition line, for example the DO line at 1083 nm in the case of helium-4.

The magnetometer also includes a circuit for exciting a plasma in the enclosure, which comprises one or more conductive elements as described above. This excitation circuit is coupled to an HF generator 4 and to overvoltage coils 5.

In one possible embodiment, electrodes are arranged on the outer surface of the enclosure to capacitively ignite and maintain a plasma discharge in the helium gas. This discharge populates the 2³S₁ state, which is the state used for magnetic measurement.

As shown in FIG. 2, these electrodes 11 can, for example, be produced by a flexible electronics process, consisting of lithography of a track of conductive material 12 (for example a layer of copper 35 microns thick) forming a pattern, for example in the shape of a fork. The tines of the fork can be dimensioned to match the outer surface of a cylindrical enclosure over its entire height. These tines are arranged on a rectangular part 13 of a flexible dielectric film, this rectangular part possibly having dimensions of 11 mm×6 mm.

Alternatively, these electrodes can be produced by depositing a thin layer of conductive material on the outer surface of the enclosure (for example a 1 micron thick layer of copper) using a physical vapour deposition process, such as evaporation or cathodic sputtering. In this case, the two electrodes are separated before the deposition is made using masking elements, such as adhesive-backed tape. Reconnection of the thin layers is also provided and can be achieved, for example, by ultrasonic micro-welding processes, or by welding onto a drop of silver lacquer.

In all these cases, the elements are connected to the radio frequency field generator 4, typically in the 10-100 MHz range. It is advantageous to have an impedance matching circuit between these electrodes and the RF generator, in order to reduce power reflections caused by excessive impedance mismatch. This circuit is produced by means of overvoltage coils 5, for example two air-core coils, with an inductance of a few tens of pH. Such impedance matching can enable the discharge to be ignited with powers of order 200 mW and maintained with powers of order 10 mW.

Once the discharge is ignited, a substantial population of atoms in the cell is brought into the 2³S₁ state, which allows magnetic measurement to be carried out in various configurations that are well known in the field, and reported, for example, in the article [5].

One of the possible operating modes of the magnetometer is to pass through the cell with a collimated laser beam, linearly polarised by means of a polariser 3 and tuned to the DO line of helium-4. This beam comes from a laser 2 with excellent amplitude stability, characterised by an RIN (Relative Intensity Noise) dominated by photon noise at powers of order 1 mW. It must also have very low phase noise. Among the laser technologies that meet this need, fibre lasers and lasers enable the best measurement performance to be achieved.

This beam is photodetected after passing through the cell. In addition, a radio frequency field, at a frequency of 40 kHz for example, is applied to the cell in a direction orthogonal to that of the polarisation of the light. This produces a signal in the photodetection spectrum at 40 kHz which, in a range of a few tens of nT around zero field, enables measurement of the magnetic field component parallel to the direction of the applied radio frequency field. Synchronous detection of this signal enables a component of the magnetic field to be measured. The intrinsic noise of the sensor then corresponds to the noise obtained at the synchronous detection output, which may come from a variety of sources. In the case described here, this noise is limited by the photon noise of the laser light used to produce the light, and corresponds in magnetic units to values between 20 and 30 fT/sqrt (Hz), which is more advantageous than the noise levels previously obtained.

The magnetometer can thus include a parametric resonance excitation circuit which includes a radio frequency generator 8 which powers Helmholtz coils 7 with orthogonal axes and which surround the cell in order to generate a parametric resonance excitation magnetic field, also referred to as a radio frequency magnetic field. The magnetometer also comprises a parametric resonance detection device 6 configured to measure the absorption of the light beam by the atomic gas, and a photodetector 10 arranged to receive the light beam having passed through the cell and to deliver a photodetection signal to said parametric resonance detection device 6.

The magnetometer can also comprise a closed-loop magnetometer control system to constantly subject the sensitive element to a total magnetic field of zero. The control system comprises a regulator 9 which is coupled to the detection device 6 and injects a current into the Helmholtz coils 7 in order to generate a compensating magnetic field Bc such that the sum Bc+B0 is maintained at zero at all times. Alternatively, the magnetometer can be operated in an open loop mode, without compensation for the ambient field.

It should be noted that since the noise is linked to the conductance of the conductive elements of the excitation circuit, it is possible to use electrodes with larger volumes than the electrodes described below but made from materials that are less good electrical conductors than copper and aluminium, for example transition metals such as Nb and Mo, alloys such as CuAl, AgCu, AuAg, or semiconductors such as Si or Ge.

In another possible embodiment, inductive ignition can be achieved using a solenoid or a helical resonator with a diameter less than or equal to 3 cm, for example, and made:
either with a copper wire, typically less than 300 microns in diameter;
or with a thicker wire made from a material with a lower conductivity than copper or aluminium, in particular made from the materials listed above for the case of capacitive electrodes.

### Comparative Examples

### Example 1a: According to the Prior Art

This example uses the cell described in [2] (a 100 mm³ cell in the form of a cylinder of 5 mm diameter and height), electrodes in the form of 4*4 mm pieces of adhesive-backed copper tape (from manufacturer 3M part number 3313, with a thickness of 35 microns) each glued to the outer surface of the cell, and connected to the RF generator by wires welded to these copper elements. The quantity Qₜₒₜₐₗ, neglecting the weld metal, is 10,700 Siemens. The unaccounted noise component resulting from measurements in low-field configuration is of order 120 fT/sqrt (Hz).

### Example 1b: According to the Prior Art

This example uses a larger cell (cylinder 1 cm in diameter and 1 cm long), also described in [2]. The electrodes in the form of 1*0.9 cm pieces of adhesive-backed copper tape (from manufacturer 3M part number 3313, with a thickness of 35 microns) each glued to the outer surface of the cell, and connected to the RF generator by wires welded to these copper elements. The quantity Qₜₒₜₐₗ, neglecting the weld metal, is 13,000 Siemens. The unaccounted noise component resulting from measurements in low-field configuration is of order 100 fT/sqrt (Hz).

### Example 2 (Prior Art)

This example uses ring electrodes such as those described in [3] and [4], each consisting of a 1 cm diameter loop made from 315 micron diameter copper wire. The quantity Qₜₒₜₐₗ is 11,700 Siemens. The unaccounted noise component resulting from measurements in low-field configuration is of the order of 77 fT/sqrt (Hz).

### Example 3 (According to the Invention)

This example uses thin-film electrodes (formed by copper deposition) of thickness 1 micron, deposited by evaporation, with masking giving the shape of a 1×1 cm rectangle on the side faces of a cylindrical cell. These electrodes are connected to the RF generator by a wire that is micro-welded by ultrasound. The quantity Qₜₒₜₐₗ, neglecting the weld metal, is 920 Siemens. The unexplained noise is less than 19 fT/sqrt (Hz), with a high uncertainty due to its lower power than that of the photon noise in the experiment (30 fT/sqrt (Hz)).

### Example 4 (According to the Invention)

This example uses thin electrodes produced on a flexible polymer such as Kapton by lithography processes that are well known in the field of flexible electronics, with a track width of 150 microns and a copper thickness of 18 microns. The quantity Qₜₒₜₐₗ is 1170 Siemens. The unexplained noise is 16 fT/sqrt (Hz), with a high uncertainty due to its low power compared with that of the photon noise during the experiment (25 fT/sqrt (Hz)).

### Example 5 (According to the Invention)

This example uses electrodes like those in Example 2, but produced with 100 micron wire. The quantity Qₜₒₜₐₗ is 1200 Siemens. The unaccounted noise component resulting from measurements in low-field configuration is of order 25 fT/sqrt (Hz).

### Example 6 (According to the Invention)

This example uses an inductive ignition system consisting of an inductive coil with a 150 micron wire wrapped 6 times around the cell with a diameter of 2 cm. The quantity Qₜₒₜₐₗ is 7900 Siemens. The unaccounted noise component resulting from measurements in low-field configuration is of order 32 fT/sqrt (Hz).

### REFERENCES

[1] W. Fourcault et al, “Helium-4 magnetometers for room-temperature biomedical imaging: toward collective operation and photon-noise limited sensitivity,” Opt. Express, vol. 29, no. 10, pp. 14467-14475, May 2021, doi: 10.1364/OE.420031.
[2] J. Rutkowski, “Study and realization of a miniature isotropic helium magnetometer,” Université de Franche Comté, 2014.
[3] M.-C. Corsi, “Helium-4 optically pumped magnetometers: development and proof of concept in magnetocardiography and magnetoencephalography,” PHD thesis, Université Grenoble Alpes, 2015. Accessed: Apr. 20, 2021. [Online]. Available: https://tel.archives-ouvertes.fr/tel-01233850
[4] FR 3 035 769 A1
[5] G. Le Gal, L.-L. Rouve, and A. Palacios-Laloy, “Parametric resonance magnetometer based on elliptically polarized light yielding three-axis measurement with isotropic sensitivity,” Appl. Phys. Lett., vol. 118, no. 25, p. 254001, June 2021, doi: 10.1063/5.0047124.

1. A cell for an optically pumped magnetometer, comprising:
an enclosure containing a gas; and
a circuit for exciting a plasma in the enclosure, which comprises one or more elements of electrically conductive material arranged so as to apply an electrical discharge to the gas contained in the enclosure,
wherein a conductance of the excitation circuit, defined as the sum of the conductance of each of the one or more elements made of electrically conductive material, is less than 8000 Siemens, the conductance of an element made of electrically conductive material corresponding to a conductivity of the electrically conductive material weighted by a ratio between a volume of electrically conductive material in said element made of electrically conductive material and a square of a distance between said element and a centre of the cell.

2. The cell according to claim 1, wherein the conductance of the excitation circuit is less than 5000 Siemens, preferably less than 1500 Siemens.

3. The cell according to claim 1, wherein the enclosure has a characteristic dimension of between 3 mm and 3 cm, preferably between 5 mm and 2 cm.

4. The cell according to claim 1, wherein the excitation circuit is capacitively coupled to the cell, the one or more of elements of electrically conductive material consisting of two electrodes.

5. The cell according to claim 4, wherein each of the two electrodes consists of a ring made of said electrically conductive material, the ring surrounding the enclosure.

6. The cell according to claim 4, wherein each of the two electrodes consists of a deposit of said electrically conductive material on the enclosure.

7. The cell according to claim 4, wherein each of the two electrodes consists of a print of said electrically conductive material on a dielectric film attached to the enclosure.

8. The cell according to claim 7, wherein the dielectric film is flexible.

9. The cell according to claim 1, wherein the excitation circuit is inductively coupled to the enclosure, the one or more elements of electrically conductive material consisting of a coil wound in a plurality of loops around the cell.

10. The cell according to claim 9, wherein the coil is a solenoid.

11. The cell according to claim 9, wherein the coil is a helix of a helical resonator.

12. An optically pumped magnetometer comprising a cell according to claim 1.

13. A magnetoencephalography helmet, comprising a plurality of optically pumped magnetometers according to claim 12.
